(19) United States
(12) Patent Application Publication
(10) Pub. No.: US 2024/0191170 A1
(43) Pub. Date: Jun. 13, 2024

(54) CULTURING APPARATUS FOR CULTURING CELL CULTURES

(71) Applicant: Charlotte OHONIN, Graz (AT)

(72) Inventors: Charlotte OHONIN, Graz (AT); Ferenc SARKÖZI, Sankt Lambrecht (AT)

(73) Assignee: Charlotte OHONIN, Graz (AT)

(21) Appl. No.: 18/286,397

(22) PCT Filed: Jul. 2, 2021
(86) PCT No.: PCT/EP2021/068285

§ 371 Date: Oct. 11, 2023

(30) Foreign Application Priority Data
Jul. 3, 2020 (AT) A 151/2020

(51) Int. Cl. C12M 1/02; C12M 1/00; C12M 1/34

(52) U.S. Cl. CPC C12M 27/00; C12M 23/40; C12M 41/00

(57) ABSTRACT

The present invention relates to a cultivation device (10) for cultivating cell cultures, having
at least one aggregation layer (20) with at least one cell input (22) for introducing a cell suspension (ZS) and at least one aggregation input (24) for introducing an aggregating fluid (AF), wherein the cell input (22) and the aggregation input (24) lead into a mixing section (26) of the aggregation layer (20) for mixing the cell suspension (ZS) and the aggregating fluid (AF) in order to form cell aggregates (ZA),
further having at least one separating layer (30) with a separating input (32) for introducing the cell aggregates (ZA) from the mixing section (26), at least one separating section (36) for separating an individual cell aggregate (ZA) from the suspension and a separating output (34) for discharging the remaining suspension after the at least one separating section (36),
further having at least one cultivation layer (40) with at least one cultivation chamber (46) with a cultivation input (42) for receiving separated individual cell aggregates (ZA) from the at least one separating section (36) and a cultivation output (44) for discharging fluid from the cultivation chamber (46).

The present invention relates to a cultivation device for cultivating cell cultures, a cultivation method for cultivating cell cultures and a cultivation station for cultivating cell cultures.

It is known that cell cultures are cultivated from individual cells in order subsequently to carry out experiments with these cultivated cells. In particular, these are human cells which are then exposed to experimental situations.

In the known solutions, cultivation of the individual cell cultures, for example in so-called Petri dishes, is usually carried out by hand. This requires as a starting point a suspension containing single cells, for example human stem cells. These stem cells can be used as a starting point to then manually create cell cultures, i.e. an aggregation of multiple individual cells. The cell aggregates which are created are then separated manually and made available accordingly for the further experiments.

A disadvantage of the known solutions is the high proportion of manual steps involved in creating the starting situation for the subsequent experiments. In particular, the selection and cultivation of the cell aggregates requires a great deal of effort. Last but not least, in the known solutions the step or location in which cultivation takes place is not identical to the location in which the subsequent experiments are carried out.

It is therefore an object of the present invention to remedy, at least in part, the disadvantages described above. In particular, it is an object of the present invention to provide, in a cost-effective and simple manner, an at least partial automation of the cultivation of cell aggregates.

The above object is achieved by a cultivation device with the features of claim 1, a cultivation method with the features of claim 13 and a cultivation station with the features of claim 16. Further features and details of the invention are disclosed in the dependent claims, the description and the drawings. Naturally, features and details described in connection with the cultivation device according to the invention also apply in connection with the cultivation method according to the invention as well as in connection with the cultivation station according to the invention and vice versa, so that with regard to disclosure mutual reference is, or can, always be made to the individual aspects of invention.

According to the invention, a cultivation device is equipped for the cultivation of cell cultures. For this purpose, such a cultivation device has at least one aggregation layer with at least one cell input for introducing a cell suspension. This aggregation layer is formed with at least one aggregation input for introducing an aggregating fluid. The cell input and the aggregate input lead into a mixing section of the aggregation layer for mixing the cell suspension and the aggregating fluid in order to form cell aggregates. In addition, the cultivation device is equipped with at least one separating layer with a separating input for introducing the cell aggregates from the mixing section of the aggregation layer. Furthermore, at least one separating section for separating an individual cell aggregate from the suspension is provided in the separating layer. Furthermore, a separating output is formed for discharging the remaining suspension after the separating section. Furthermore, the cultivation device is equipped with at least one cultivation layer with at least one cultivation chamber with a cultivation input for receiving separated individual cell aggregates from the at least one separating section. In addition, a cultivation output for discharging fluid from the cultivation chamber is provided in the cultivation layer.

In the context of the present invention, cell cultures are to be understood to mean both 2D and 3D structures, in particular tissue structures, for example in the form of spheroid structures or organoid structures.

A core idea behind the invention is based on automating at least part of the cultivation of the cell cultures or even carrying out this cultivation step completely automatically. In the following it will be explained how the cultivation can be carried out using the three layers.

In a first step, the cell suspension and the aggregating fluid are brought together in the aggregation layer. The cell suspension is a fluid in which a plurality of individual cells are arranged. These individual cells can also already be partially aggregated. In the mixing section of the aggregation layer, the aggregating fluid mixes with this cell suspension and in this way forms an aggregation of the cell aggregates, so that agglomerates can form from individual cells in the form of the cell aggregates. Mixing the aggregating fluid and cell suspension thus involves a first step for the subsequent cultivation, namely the aggregation of individual cells to form cell aggregates.

Within the context of the present invention, different possibilities exist for the above aggregation. For example, in one embodiment of the present invention, the cell suspension may contain a gelling agent, in particular a gelling liquid, for example in the form of alginate. This mixture is introduced into the mixing section through the cell input as a cell suspension. In this embodiment, aggregating fluid is introduced via the aggregation input in the form of a saline solution, for example containing a chlorine salt. On being mixed in the mixing section, the chlorine salt activates the gelling liquid, so that crystalline structures form around the cell aggregates, which can then be separated from each other in the separating layer.

Alternatively, in a further embodiment, aggregation by means of an oil-containing aggregating fluid is conceivable in the context of the present invention. In this case, the cell suspension can be introduced into the mixing section via the cell input without further aggregation additive. The oil-containing aggregating fluid, for example containing a nut oil, can be fed into the mixing section via the aggregation input, bringing about the aggregation of the cell aggregates through hydrophobic and hydrophilic interactions.

Naturally, other mechanisms based on chemical, biological and/or physical interactions are conceivable for the aggregation of the cell aggregates. According to the present invention, a suitable cell suspension and a suitable aggregating fluid is selected depending on the selected aggregation function.

According to the invention, the subsequent step of separating the individually formed cell aggregates is carried out in the cultivation device in the following separating layer. The formed cell aggregates are transferred from the mixing section to this separating layer via the separating input. The separating section is in particular a geometrically and/or mechanically acting separating section which has a smaller passage dimension than the desired size of the cell aggregates formed, creating a sieve effect. In other words, fluid containing the formed cell aggregates is now transferred from the mixing section to the separating section. This can be done, for example, by forced delivery using pumping means. When the cell aggregates move into the separating section, they remain, individually separated, in the respective separating sections. It is important that, in a separating section according to the invention, this is specifically designed for an individual cell aggregate. This separating section thus serves to separate an individual cell aggregate from a quantity of formed cell aggregates. At the end of this separation step, one or more provided separating sections are filled with correspondingly individually separated cell aggregates.

In the final step, the individually separated cell aggregates are in each case transferred to an associated cultivation chamber. For this purpose, each cultivation chamber is connected with a corresponding separating section, in a fluid-communicating manner, via a cultivation input. This allows the individually separated cell aggregates to be introduced specifically into a cultivation chamber provided for this purpose and to be further cultivated there and/or to be used for subsequent test series.

As can be seen from the preceding explanation of the procedure, the formation of the cell aggregates in the aggregation layer, the separation into individual cell aggregates in the separating layer and then the transfer to a cultivation chamber in the cultivation layer can now be carried out without external intervention. As can also be seen, the cultivation chamber, as a location for individually separated cell aggregates, is now able to serve for further cultivation of these cell aggregates. Thus, it is also possible to introduce other media, for example nutrient media for the further growth of the introduced individual cell aggregates, via the cultivation input. It is therefore possible, after introducing the cell aggregates, to cultivate these in the respective cultivation chamber, to supply these with nutrients and in this way to allow these to continue to grow.

As a result, the cultivation device provides a biological chip, whereby a correspondingly cultivated cell aggregate is preferably present in each individual cultivation chamber, of which at least one is provided in the cultivation layer. This biochip can now be used in biological, chemical and/or pharmaceutical experiments. For example, it is possible, via the cultivation inputs, to supply the cultivation chamber and thus the cell aggregates arranged therein not only with nutrient fluid for cultivation, but also with sample fluid for carrying out an experimental set-up. In order to further explain or clarify this, an example is described in the following.

For example, it is conceivable that an organ is to be simulated from human stem cells on a biochip according to the invention for subsequent pharmaceutical test series. For example, liver stem cells can be used, in the described manner, to combine these to form cell aggregates in the aggregation layer, to separate these into individual cell aggregates in the separating layer and then to transfer these, in the individually separated form, into the cultivation chambers. In the subsequent step, cultivation leads to a cell aggregate consisting of numerous cells being able to grow in each individual cultivation chamber of the at least one cultivation layer, which accordingly corresponds to an aggregation of human liver cells. In a subsequent experimental step, it is now possible to supply each of these cultivation chambers and thus each of these cell aggregates, which can represent a human liver for the test series, with appropriate sample fluids and to measure the reaction to these sample fluids. Such sample fluids can for example be pharmaceutical compositions whose effect on liver cells are to be tested. As can be seen from this explanation, not only can an automated provision of individual cell aggregates be achieved in a simple and cost-effective manner, the subsequent performance of biological, pharmaceutical and/or chemical experiments can also be facilitated and/or automated. It is therefore possible to cultivate cell aggregates with a high throughput and with a high degree of automation and then, using a cultivation device according to the invention, to tailor the cultivated cell aggregates to the desired experimental set-ups.

It should also be noted that the individual layers, i.e. the at least one aggregation layer, the at least one separating layer and the at least one cultivation layer, are connected to each other in a fluid-communicating manner, in particular the corresponding inputs, outputs and sections, as have been explained several times. In the simplest form, these fluid-communicating connections between the individual layers correspond to simple correlating openings. As will be explained later, these layers may in particular be formed as flat or substantially flat layers, which may, in a simple and inexpensive manner, be arranged on top of each other, at least partially overlapping.

The formation of individual layers, which as described in the preceding paragraph can be arranged overlapping on top of each other, means that it is also possible to manufacture such a cultivation device in a particularly simple and cost-effective manner. Thus, a manufacturing process for producing such a cultivation device according to the invention in which, in a first step, a separating layer with the desired fluid-communicating connection between the mixing section and the separating input is arranged on an aggregation layer, is also a part and object of the present invention. Finally, at least one cultivation layer is arranged on the separating layer, wherein the cultivation input of the at least one cultivation chamber is connected in a fluid-communicating manner with a separating section with the associated possibility of separating individual cell aggregates. Thus, such a manufacturing process also brings with it the advantages which have been explained or will be explained with reference to a cultivation device according to the invention.

It may bring advantages if, in a cultivation device according to the invention, the at least one aggregation layer, the at least one separating layer and/or the at least one cultivation layer are formed from physically separated materials. This makes it possible to produce the individual layers in a cost-effective and simple way. The physical separation of the materials is in particular based on the fact that identical or substantially identical materials can be used for each of the individual layers. For example, it is conceivable that a plastic material is provided for the individual layers. If the individual layers are at least partially placed on top of each other, they can have a planar contact surface with each other, which at the same time can also form the desired sealing functionality for sealing the individual fluid-carrying sections within the layers.

Further advantages can be achieved if, in a cultivation device according to the invention, the at least one aggregation layer, the at least one separating layer and/or the at least one cultivation layer have planar, in particular flat or substantially flat, base bodies. These planar, flat base bodies mean that production can be made even more simple and cost-effective. For example, it is possible to provide the individual layers of a material which can be processed by photoactive influence or by chemical influence, for example in the form of an etching agent. This makes it possible to form cavities or openings which can form the fluid-carrying sections, inputs and outputs in the desired manner. It is possible that the individual fluid-carrying components of the layers represent both closed channels, channels open at one end or complete openings through the material of the respective layer. In channels which are open at one end or completely open, the corresponding sealing can preferably be provided by the planar contact with the layer arranged above and/or beneath.

It is also advantageous if, in a cultivation device according to the invention, the cell input, the aggregation input, the mixing section, the separating input, the separating section, the separating output, the cultivation input, the cultivation chamber and/or the cultivation output are formed as a material recess in the aggregation layer, the separating layer and/or the cultivation layer. As already explained in the previous paragraph, this can be provided for example by photoactive means and material combinations, etching techniques or the like. Techniques such as cutting plotting, injection moulding, laser cutting or the like can also be used. Naturally, the individual layers can also be composed of partial layers in order to provide correspondingly larger flow cross-sections for the individual inputs, outputs and sections. The channels have associated fluid interfaces, as can be provided in the simplest way as openings between the individual inputs, outputs and sections or between the layers.

It is also advantageous if, in a cultivation device according to the invention, the at least one aggregation layer, the at least one separating layer and/or the at least one cultivation layer are, at least in sections, arranged on top of each other. This arrangement on top of each other, especially in direct, preferably planar contact, leads to a particularly space-saving arrangement. Manufacture is also significantly simplified, since the sealing of the individual layers with respect to each other, and thus the sealing of the fluid-carrying sections with respect to each other, can preferably be provided through this planar contact. With the same dimensions and with simplified manufacture, such a cultivation device can thus be made available with a maximum reduction in space.

It is also advantageous if, in a cultivation device according to the invention, the at least one aggregation layer, the at least one separating layer and/or the at least one cultivation layer have at least one continuous fluid channel running transversely to their main directions of extension. The main directions of extensions can for example be the planar extensions, in the case of planar, substantially flat layers. The orientation of a corresponding fluid channel transversely to this main direction of extension leads to such a fluid channel preferably extending through two or more such layers. For example, where fluid-carrying sections, inputs and outputs are arranged along the main direction of extension of the individual layers, this makes it possible to provide a supply and a removal of corresponding fluids from above and/or below this cultivation device, transversely thereto. This makes possible a facilitated supply as well as a simplified arrangement in a cultivation device of corresponding connections to external reservoir containers. These one or more fluid channels thus make possible a supply or removal of the fluids for transverse distribution within the respective layer.

It is further advantageous if, in a cultivation device according to the invention, an upper and/or a lower sealing layer form a seal. This sealing layer thus forms an additional layer to the aggregation layer, the separating layer and/or the cultivation layer. In particular, if the individual functional layers are particularly thin, for example in the case of complete openings for the individual fluid-carrying sections, a separate sealing layer is advantageous. It makes it possible to achieve the formation of the individual functional layers even more cost-effectively and simply. It is also conceivable that several layer arrangements are arranged parallel to each other, in a sandwich construction, these being sealed by a common sealing layer above and/or below. This makes it possible to produce, in a cost-effective and simple way, a cultivation device with numerous partial cultivation sections arranged side by side with a common sealing layer at the top and bottom. In this way, interfaces between individually cultivated cell aggregates can be provided, also allowing interactions between cell aggregates to be examined.

It is also advantageous if, in a cultivation device according to the invention, valve means are arranged between the individual layers for blocking and releasing the individual fluid flows. In principle, it is conceivable that externally supplied fluid flows and corresponding opening and closing of externally arranged valves provide the desired functionality. However, it is also conceivable in principle for internal valve means to be provided in order to improve control and/or in order to avoid dead spaces or in order to carry out a targeted rinsing process. Such valve means can be switchable valve means, but also pressure-sensitive automatically switched valve means.

Further advantages can be achieved if, in a cultivation device according to the invention, a common collective outlet is formed as cultivation output for all cultivation chambers. This makes it possible, in a net-like structure consisting of several cultivation chambers, to discharge the waste materials and the waste fluid together, and via a single common fluid connection acting as a collective outlet, as cultivation output. In particular, such a cultivation output opens into a fluid channel arranged transversely to the main extension of the cultivation layer, which allows a cost-effective and simple removal of the waste fluid.

It is further advantageous if, in a cultivation device according to the invention, the aggregation layer has at least two cell inputs and/or at least two aggregation inputs. In particular, the individual cell inputs and/or the individual aggregation inputs are equipped with equal or substantially equal flow diameters to provide the same flow conditions. Depending on which mixing ratios and which mixing parameters are desired, different ratios of cell inputs to aggregation inputs can be provided. Naturally, individual cell inputs and/or individual aggregation inputs can also be switched off for corresponding implementation or for corresponding application situations.

It also brings advantages if, in a cultivation device according to the invention, the at least one separating section is designed to be switchable and/or variable. Such variation is possible, for example, in the manufacture of the cultivation device. Thus, depending on the size of the desired cell aggregates which are to be individually separated, a geometrically larger or a geometrically smaller passage can be provided for the respective separating section. However, in principle it is also possible that a separating section is already designed to be switchable in a finished cultivation device, for example so that it can vary its passage geometry. If, for example, an electrical voltage is applied, with a correspondingly suitable selection of the material of the separating section this can increase and/or decrease its passage geometry, so that it becomes possible to switch or vary an adaptation to the actually desired separation size of the cell aggregates, even with an existing cultivation device.

Further advantages can be achieved if, in a cultivation device according to the invention, at least one measuring device is arranged, in particular in the cultivation layer, for a measurement of chemical and/or physical parameters. Such a measuring device allows, for example, parameters to be determined during an experimental set-up and/or during cultivation. This can for example be the concentration of particular chemicals, temperatures, pressures, pH values, pOH values, photometric determinations or the like. Such a measuring device can also be a combined measuring device for determining two or more different parameters. The measurement can be used both for cultivation operation and also for the experimental operation carried out later in the same cultivation device.

The subject matter of the present invention also includes a cultivation method for cultivating cell cultures using a cultivation device according to the invention. Such a cultivation method comprises the following steps:
introducing cell suspension and aggregating fluid into the mixing section of the cultivation device in order to form cell aggregates,
transporting the formed cell aggregates into the separating section in order to separate individual cell aggregates,
transporting the separated individual cell aggregates into a respective cultivation chamber.

A cultivation method according to the invention thus brings the same advantages as have been explained in detail with reference to a cultivation device according to the invention. The functionality of the individual steps has also already been explained in detail in the introduction to these application documents. Following transfer into the cultivation chambers, the transported cell aggregates are supplied with supply medium.

It brings advantages if, in a cultivation method according to the invention, a rinsing of the mixing section and/or the separating section takes place before the step of transfer into the cell chambers and/or afterwards. This makes it possible to flush out the remaining suspension and/or the remaining aggregating fluid in order in this way to make possible a subsequent cultivation operation, for example the supply of nutrient solution, and/or a subsequent experimental operation and thus a supply with appropriate test fluid.

Further advantages can be achieved if, in a cultivation method according to the invention, a supply of the cultivation chambers with nutrients and/or test fluids takes place after the individual cell aggregates are transferred into the cultivation chambers. The supply of nutrients means that the individual cell aggregates can grow in the cultivation chambers. Operation for carrying out an experimental set-up is carried out with supply of nutrients and/or test fluids so that, for example, pharmaceutical test fluids are introduced into the cultivation chambers in order to observe the reaction of the cell aggregates cultivated therein.

The subject matter of the present invention also includes a cultivation station for cultivating cell cultures, comprising at least one cultivation device according to the present invention. This cultivation station further comprises at least one reservoir for storing at least one of the following fluids:
cell suspension,
aggregating fluid,
nutrient fluid,
test fluid.

The above list is a non-exhaustive list. A cultivation station according to the invention brings the same advantages as have been explained in detail with reference to a cultivation device according to the invention. In addition to storing the individual fluids, it is also conceivable that one or more injection inputs are provided in order to provide a single or multiple injection of a fluid or different fluids in individual operating situations. Delivery devices, for example pumping means, are also preferably provided to transport the individual fluids to the desired inputs of the cultivation device. In addition, it is possible to provide one or more control modules to control valve means and/or such delivery devices. Last but not least, such a control module can provide appropriate connectivity for data synchronisation with a cloud or a downstream local data evaluation unit.

It is advantageous if, in a cultivation station according to the invention, at least two cultivation devices according to the present invention are provided and are connected to each other, at least in sections, in a fluid-communicating manner. This cross-linking of the fluid-communicating connection makes it possible to examine significantly more complex questions in experimental set-ups. For example, a first cultivation device may represent one or more cell aggregates of a first human organ and the correspondingly adjacent cross-linked cultivation device a plurality of cell aggregates of a second human organ. This makes it possible also to take into account a cross-connection between the organs and thus a cross-correlation in an experimental set-up. In particular, individual cultivation chambers of the first cultivation device are connected in a fluid-communicating manner with correlating cultivation chambers of the second cultivation device. Such a connection can also include a biomembrane, which is in particular designed to be semipermeable. Extracellular matrix proteins, e.g. collagen, can also be arranged on such a biomembrane for improved interaction with the respective adjacent cell culture.

Further advantages, features and details of the invention are explained in the following description, in which exemplary embodiments of the invention are described in detail with reference to the designations. The features mentioned in the claims and in the description may in each case be essential to the invention individually or in any combination. In each case schematically:

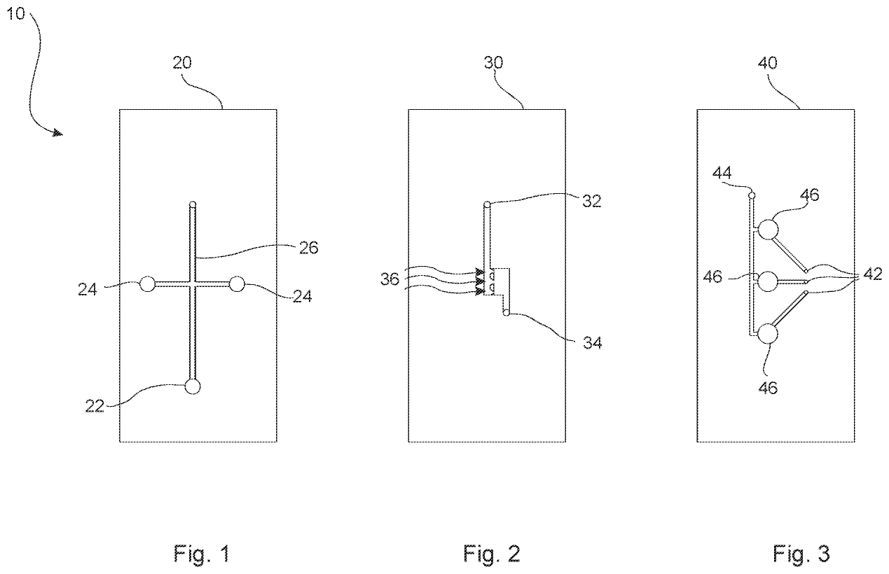

FIG. 1 shows an embodiment of an aggregation layer,

FIG. 2 shows an embodiment of a separating layer,

FIG. 3 shows an embodiment of a cultivation layer,

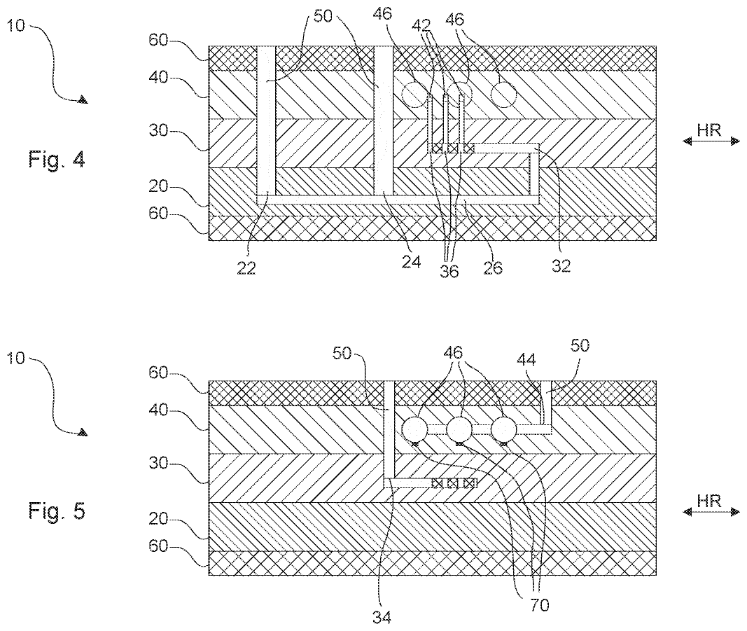

FIG. 4 shows a cross-section through a cultivation device according to the invention,

FIG. 5 shows a cross-section along another sectional plane according to FIG. 4,

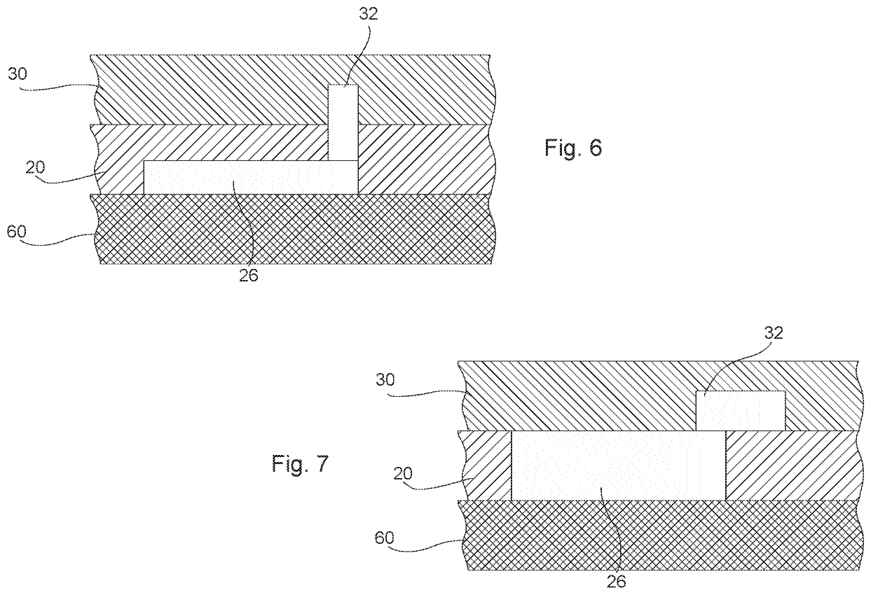

FIG. 6 shows a detail representation of a cultivation device,

FIG. 7 shows another detail representation of a cultivation device,

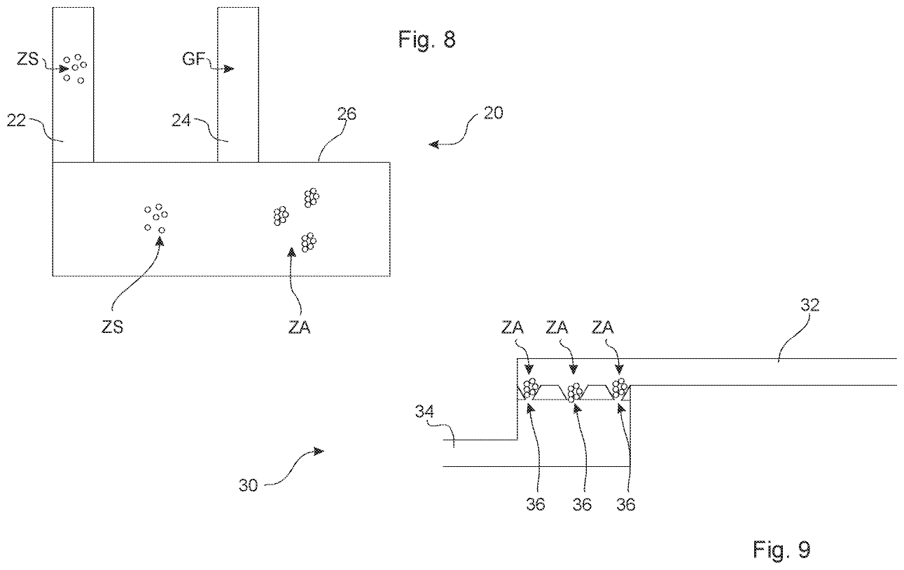

FIG. 8 shows a first step of a method according to the invention,

FIG. 9 shows a further step of a method according to the invention,

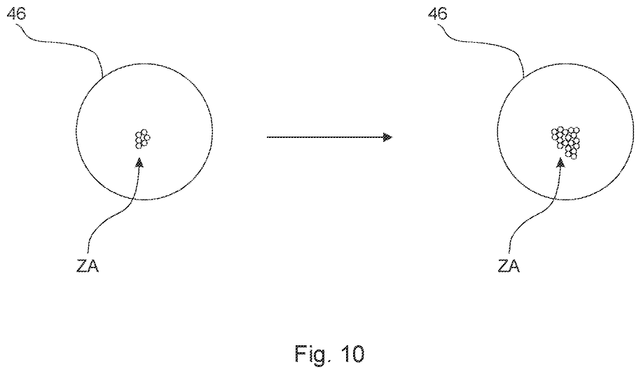

FIG. 10 shows a further step of a method according to the invention,

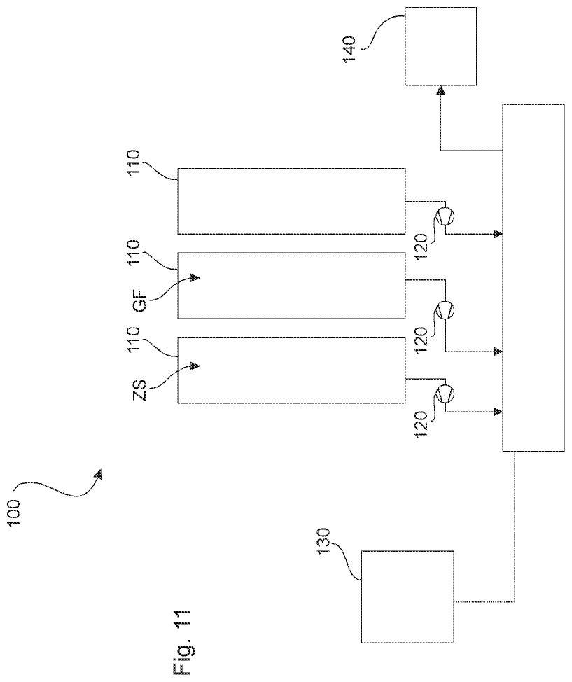

FIG. 11 shows an embodiment of a cultivation station according to the invention.

A plan view of different layers is shown schematically in FIGS. 1 to 3. In this cultivation device 10, an aggregation layer 20 is shown in FIG. 1 which in this case has two aggregation inputs 24 running perpendicular to the drawing plane. A cell input 22 is also provided in this aggregation layer 20. The aggregation inputs 24 and the cell input 22 open into a common mixing section 26 in order to mix cell suspension ZS and aggregating fluid AF.

FIG. 2 shows that the formed cell aggregates ZA can be transferred to the separating layer 30 from the mixing section 26 of the aggregation layer 20 via a separating input 32. In this case, by way of example, three separating sections 36 are provided here which can accordingly collect three cell aggregates ZA and separate these individually. The fluid flowing onwards in the form of the free suspension can be discharged into the environment or collected via the separating output 34. This separating output 34 forms a bypass downstream of the separating section 36, so that further cell aggregates ZA are transported further via this bypass after the separation section 36 has been occupied.

FIG. 3 shows that each of the individual separating sections 36 is connected in a fluid-communicating manner with one of the three cultivation chambers 46 arranged in the cultivation layer 40 via its own cultivation input 42. The separated cell aggregates ZA from the individual separating sections 36 can in each case be transferred individually and specifically into a cultivation chamber 46. In order to remove suspension and superfluous fluid, this can be discharged into the environment or collected via a cultivation output 44.

FIGS. 4 and 5 show that the individual layers 20, 30 and 40, as shown for example in FIGS. 1 to 3, can be flat or planar. In such a flat embodiment, the individual layers 20, 30 and 40 can be superimposed, wherein they may be correlated with each other in cross-section as shown in FIGS. 4 and 5. In FIG. 4 it is shown that the cell input 22 and the aggregation input 24 can be supplied via fluid channels 50 running vertically, i.e. transversely to the main direction of extension HR. The cell aggregates from the mixing section 26 can also be transferred to the separating input 32 of the separating layer 30 via a fluid communication likewise running transversely to the main direction of extension HR. The fluid communication from the separating layer 30 to the cultivation layer 40 also runs transversely to the main direction of extension HR. FIG. shows a cross-section along another plane and in this way the separating output 34, also shown as the vertical fluid channel 50, and the cultivation output 44 can be seen.

FIG. 6 shows schematically that, by way of example, in the aggregation layer 20, the mixing section 26 can be designed as a partial cut-out or partial recess in the material. In order to provide a sealing of this mixing section 26, a lower sealing layer 60 is provided here, which provides the desired sealing. As well as a partial cut-out as shown in FIG. 6, a complete removal of material as shown in FIG. 7 is also conceivable, so that the mixing section 26 can be understood as an opening through the aggregation layer 20. FIGS. 6 and 7 also show the fluid-communicating communication to the separating input 32 in the separating layer 30 for the different solutions.

A method according to the invention can be explained in more detail with reference to FIGS. 8, 9 and 10. Thus, FIG. 8 is a representation of a first step in which cell suspension ZS with individual cells is introduced into the mixing section 26 via the cell input 22. The aggregating fluid AF is introduced via the aggregation input 24, so that cell aggregates ZA are formed in the mixing section 26. FIG. 9 shows how these cell aggregates ZA can be filtered out, so to speak, since they do not fit geometrically through the individual separating sections 36. FIG. 9 shows the situation of three filled separating sections 36, in which the cell aggregates ZA are present, in each case individually separated. From this separated position shown in FIG. 9, it is now possible to transfer individual cell aggregates ZA into a cultivation chamber 46 and allow them to grow there, as the time delay of FIG. 10 shows. The grown and cultivated cell aggregates ZA can then be used as a basis for an experimental set-up.

FIG. 11 shows a cultivation station 100 which in this case has four reservoirs as reservoirs 110. In two reservoirs 110, cell suspension ZS and aggregating fluid AF is provided. More fluid, for example, may be introduced into another reservoir 110. It can also clearly be seen here that pumps are provided as delivery means 120 to supply the cultivation device 10 arranged below. Also, a control module 130 can control or regulate the individual delivery means 120, but also corresponding valves. A collecting vessel 140 makes it possible to collect the fluid issuing from the cultivation device.

The above explanation of the embodiments describes the present invention exclusively with reference to examples. Naturally, individual features of the embodiments can, if technically expedient, be freely combined with each other without departing from the scope of the present invention.

### LIST OF REFERENCE SIGNS

10 cultivation device
20 aggregation layer
22 cell input
24 aggregation input
26 mixing section
30 separating layer
32 separating input
34 separating output
36 separating section
40 cultivation layer
42 cultivation input
44 cultivation output
46 cultivation chamber
50 fluid channel
60 sealing layer
70 measuring device
100 cultivation station
110 reservoir
120 conveying means
130 control module
140 collecting vessel
ZS cell suspension
AF aggregating fluid
ZA cell aggregates
HR main direction of extension

1. Cultivation device (10) for cultivating cell cultures, having
at least one aggregation layer (20) with at least one cell input (22) for introducing a cell suspension (ZS) and at least one aggregation input (24) for introducing an aggregating fluid (AF), wherein the cell input (22) and the aggregation input (24) lead into a mixing section (26) of the aggregation layer (20) lead for mixing the cell suspension (ZS) and the aggregating fluid (AF) in order to form cell aggregates (ZA),
further having at least one separating layer (30) with a separating input (32) for introducing the cell aggregates (ZA) from the mixing section (26), at least one separating section (36) for separating an individual cell aggregate (ZA) from the suspension and a separating output (34) for discharging the remaining suspension after the at least one separating section (36),
further having at least one cultivation layer (40) with at least one cultivation chamber (46) with a cultivation input (42) for receiving separated individual cell aggregates (ZA) from the at least one separating section (36) and a cultivation output (44) for discharging fluid from the cultivation chamber (46).

2. Cultivation device (10) according to claim 1, wherein the at least one aggregation layer (20), the at least one separating layer (30) and/or the at least one cultivation layer (40) are formed from physically separated materials.

3. Cultivation device (10) according to claim 1, wherein the at least one aggregation layer (20), the at least one separating layer (30) and/or the at least one cultivation layer (40) have planar, in particular flat or substantially flat, base bodies (21, 31, 41).

4. Cultivation device (10) according to claim 1, wherein the cell input (22), the aggregation input (24), the mixing section (26), the separating input (32), the separating section (36), the separating output (34), the cultivation input (42), the cultivation chamber (46) and/or the cultivation output (44) are formed as a material recess in the aggregation layer (20), the separating layer (30) and/or the cultivation layer (40).

5. Cultivation device (10) according to claim 1, wherein the at least one aggregation layer (20), the at least one separating layer (30) and/or the at least one cultivation layer (40) are, at least in sections, arranged on top of each other.

6. Cultivation device (10) according to claim 5, wherein the at least one aggregation layer (20), the at least one separating layer (30) and/or the at least one cultivation layer (40) have at least one continuous fluid channel (50) running transversely to their main extension directions (HR).

7. Cultivation device (10) according to claim 1, wherein an upper and/or a lower sealing layer (60) form a seal.

8. Cultivation device (10) according to claim 1, wherein valve means are arranged between the individual layers (20, 30, 40) for blocking and releasing the individual fluid flows.

9. Cultivation device (10) according to claim 1, wherein a common collective outlet is formed as cultivation output (44) for all cultivation chambers (46).

10. Cultivation device (10) according to claim 1, wherein the aggregation layer (20) has at least two cell inputs (22) and/or at least two aggregation inputs (24).

11. Cultivation device (10) according to claim 1, wherein the at least one separating section (36) is designed to be switchable and/or variable.

12. Cultivation device (10) according to claim 1, wherein at least one measuring device (70) is arranged, in particular in the cultivation layer (40), for a measurement of chemical and/or physical parameters.

13. Cultivation method for cultivating cell cultures with a cultivation device (10) with the features of claim 1, comprising the following steps:
introducing cell suspension (ZS) and aggregating fluid (AF) into the mixing section (26) of the cultivation device (10) in order to form cell aggregates (ZA),
transporting the formed cell aggregates (ZA) into the separating section (36) in order to separate individual cell aggregates (ZA),
transporting the separated individual cell aggregates (ZA) into a respective cultivation chamber (46).

14. Cultivation method according to claim 13, wherein a rinsing of the mixing section (26) and/or the separating section (36) takes place before the step of transfer into the cell chambers (46) and/or afterwards.

15. Cultivation method according to claim 13, wherein after the individual cell aggregates (ZA) are transferred into the cultivation chambers (46) a supply of the cultivation chambers (46) with nutrients and/or with test fluids takes place.

16. Cultivation station (100) for cultivating cell cultures, comprising at least one cultivation device (10) with the features of claim 1, further comprising at least one reservoir (110) for storing at least one of the following fluids:
cell suspension (ZS)
aggregating fluid (AF)
nutrient fluid
test fluid.

17. Cultivation station (100) according to claim 16, wherein the at least one cultivation device (10) is one of wherein at least two cultivation devices (10) of the Cultivation station which are connected to each other, at least in sections, in a fluid-communicating manner.
